(19) United States
(12) Patent Application Publication
(10) Pub. No.: US 2025/0195515 A1
(43) Pub. Date: Jun. 19, 2025

(54) COMPOSITION FOR TREATING THE EYE

(71) Applicant: BITOP AG, Witten (DE)

(72) Inventors: Andreas BILSTEIN, Bergheim (DE); Hans-Joachim GALLA, Munster (DE); Mridula DWIVEDI, Munster (DE)

(73) Assignee: BITOP AG, Witten (DE)

(21) Appl. No.: 19/006,532

(22) Filed: Dec. 31, 2024

Related U.S. Application Data
(63) Continuation of application No. 17/395,320, filed on Aug. 5, 2021, now abandoned.
Application No. 17/395,320 is a continuation of application No. 15/312,970, filed on Nov. 21, 2016, now abandoned.

(30) Foreign Application Priority Data
May 22, 2014 (DE) 102014007423.4

(51) Int. Cl. A61K 31/505; A61K 9/00; A61K 9/127

(52) U.S. Cl. CPC A61K 31/505; A61K 9/0048; A61K 9/127; A61K 2121/00

(57) ABSTRACT

AMENDMENTS TO THE ABSTRACT

The invention relates to a composition containing, as active ingredient, ectoine, hydroxyectoine and/or salts, esters or amides of these compounds for the treatment and/or prophylaxis of diseases of the eye that are associated with a disturbance of the tear film. The disease can be in particular keratoconjunctivitis sicca. It has been found that, surprisingly, these compounds are capable of preventing a rupture in the outer lipid layer of the tear film, avoiding an undesirably rapid evaporation of the lacrimal fluid.

The present application is a continuation of U.S. patent application Ser. No. 17/395,320, filed Aug. 5, 2021, which is a continuation of U.S. patent application Ser. No. 15/312,970, filed Nov. 21. 2016, now abandoned, which is a National Stage of International Application No. PCT/EP2015/061434, filed May 22, 2015, which claims priority to German patent application no. 102014007423.4, filed May 22, 2014, the entirety of all of which are hereby incorporated by reference.

### FIELD

The invention relates to a composition which serves for the treatment of diseases of the eye that are associated with a disturbance of the tear film, especially dry eye syndrome (keratoconjunctivitis sicca).

### BACKGROUND

The lacrimal glands (glandulae lacrimales) in humans are responsible for the production of lacrimal fluid. Said fluid is a clear, slightly alkaline fluid which serves for moistening the conjunctiva and the cornea. Disturbances in the production of tears or in the composition of the tear film can lead to dry eye syndrome (keratoconjunctivitis sicca, keratitis sicca; DES). Symptoms associated therewith are a foreign body sensation, burning sensation and red eyes. In severe cases, corneal damage right up to loss of sight can occur. Keratoconjunctivitis sicca is a commonly occurring disease affecting approx. from 10 to 20% of the adult population. Treatment is frequently carried out with hyaluronic acid, artificial lacrimal fluid or cellulose derivatives. However, said treatment is frequently unsatisfactory owing to inadequate treatment outcome or adverse effects.

Besides the lacrimal glands, the meibomian glands (glandulae tarsales) are of importance. Here, the glands are sebaceous glands at the rim of the eyelids. The meibomian glands release an oily fluid which mixes with the lacrimal fluid. Overall, an outer lipid layer arises on the tear film, causing the aqueous lacrimal fluid not to evaporate too rapidly. Disturbances in the secretion process of the meibomian glands, for example in the lipid composition of the secretion fluid, or a rupture in the lipid layer, therefore lead to premature evaporation of the lacrimal fluid and thus facilitate the development of a keratoconjunctivitis sicca.

The object is therefore to provide a composition which prevents a rupture in the lipid layer, associated with increased evaporation of lacrimal fluid and hence the occurrence of eye diseases associated therewith. More particularly, it is intended that the composition prevent the development of a keratoconjunctivitis sicca or treat it.

### SUMMARY

According to the invention, this object is achieved by a composition containing, as active ingredient, ectoine, hydroxyectoine and/or salts, esters or amides of these compounds.

Ectoine and hydroxyectoine are tetrahydropyrimidine derivatives which are synthesized under stress conditions by extremophilic microorganisms, more particularly halophilic microorganisms. To date, various uses have been described for ectoine and hydroxyectoine, for example as moisturizer, for the treatment of vascular leak syndrome (VLS) (DE 10 2006 056 766 Al) or for the treatment of neurodermatitis (DE 103 30 243 Al). DE 100 06 578 A1 discloses the use of ectoine and its derivatives to protect biopolymers from degradation by degrading enzymes such as proteases, nucleases or lipases.

The systematic name for ectoine is 2-methyl-1,4,5,6-tetrahydropyrimidine-4-carboxylic acid, and for hydroxyectoine it is 5-hydroxy-2-methyl-1,4,5,6-tetrahydropyrimidine-4-carboxylic acid.

The structure of the natural L-ectoine ((S)-2-methyl-1,4,5,6-tetrahydropyrimidine-4-carboxylic acid) is depicted below:

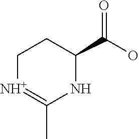

The structure of the natural hydroxyectoine ((4S,5S)-5-hydroxy-2-methyl-1,4,5,6-tetrahydropyrimidine-4-5 carboxylic acid) is displayed below:

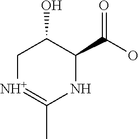

The use of the specified stereoisomers is preferred, 10 but not obligatory, i.e., the use of other stereoisomers or of the racemate is possible too.

The tear film serving for moistening and protecting conjunctiva and cornea essentially consists of an inner mucin-rich layer, an aqueous phase containing proteins, metabolites and salts, and also the outer lipid layer at the interface between liquid and air. The lipid layer is composed of various lipids, more particularly polar phospholipids and fatty acids, cholesteryl esters and triglycerides. In this connection, the polar lipids form the bottom part of the lipid layer, i.e., they form the boundary to the underlying aqueous phase. Cholesteryl esters and other sterol esters are embedded between the nonpolar head groups of the polar lipids, and so a kind of platform is formed for the nonpolar portions of the lipid layer that are situated further out, more particularly the triglycerides.

The invention is based on the finding that one of the reasons for a premature rupture in the lipid layer is explained by its increased rigidity in keratoconjunctivitis sicca patients. In the individuals affected, the ratio of neutral to polar lipids is increased. Accordingly, it was possible to establish an increased lipid-lipid interaction in comparison with healthy individuals. The associated increased rigidity of the lipid layer can lead to the formation, in said layer, of gaps through which the lacrimal fluid comes into contact with the air and evaporates to a very great extent.

The composition according to the invention is especially suited for treating keratoconjunctivitis sicca, but can also be used in other eye diseases which are associated with a disturbance of the tear film, for example for treating blepharitis or meibomitis. In general, the composition is suited for treating a xerophthalmia, in which a distinction is made between the hyperevaporative form, characterized by a dysfunction of the meibomian glands, and the hypovolemic form, distinguished by insufficient lacrimation. The composition is particularly suited for treating dry eye syndrome in its hyperevaporative form.

It has now been found that ectoine and hydroxyectoine are capable of ensuring a fluidization of the lipid layer and of thus preventing a keratoconjunctivitis sicca or treating it. The composition according to the invention is therefore capable of remedying disturbances associated with the lipid layer generated by the meibomian glands, for example with respect to the composition. Specifically, the area occupied by the individual phospholipids increases. This brings about a reduced order and larger gaps in the phospholipid layer and thus less platform for the hydrophobic constituents of the lipid layer, more particularly the triglycerides. Since these have less space available, small droplet-type structures composed of triglycerides can be formed on the outer lipid layer. Altogether, the lipid layer becomes more mobile and less rigid; the probability of rupturing in the lipid layer is reduced. Furthermore, spreading of the lipid layer is facilitated, and the elasticity of the lipid layer is increased: especially in the event of exertion of pressure, some lipids, especially the nonpolar triglycerides, can converge to form droplet-type structures, and in the event of expansion, these can spread out again in order to ensure a stable lipid layer.

### BRIEF DESCRIPTION OF THE DRAWINGS

Exemplary embodiments of the invention will be described hereinbelow with reference to the figures, in which:

FIG. 1A: FIG. 1A depicts an example of the tear film without addition of ectoine, with 1 showing the aqueous phase;

FIG. 1B: FIG. 1B depicts the example embodiment of FIG. 1A in the presence of 100 mM ectoine in the lacrimal fluid;

FIG. 2A: FIG. 2A depicts compression of a lipid film on a PBS subphase without ectoine;

FIG. 2B: FIG. 2B illustrates compression-expansion curves, with respect to hysteresis;

FIG. 2C: FIG. 2C illustrates the compression-expansion curves of FIG. 2B, wherein the presence of ectoine is concentration-dependent;

FIG. 3: FIG. 2C depicts how the droplet-type structures are more fluid than the other regions of the lipid layer, indicative of a concentration of triglyerides which are less strongly molecularly organized than phospholipids and cholesteryl esters.

### DETAILED DESCRIPTION

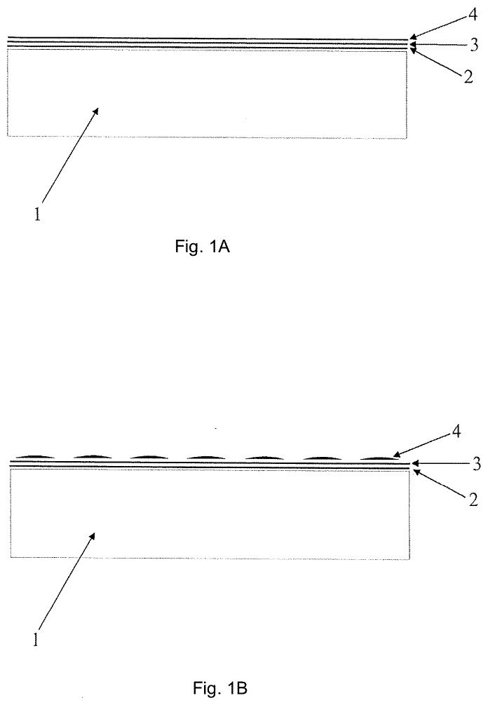

A macroscopic model of the effect which a sufficiently high ectoine concentration exerts on the tear film is displayed schematically in FIG. 1A shows the tear film without addition of ectoine, with 1 showing the aqueous phase. The boundary layer 2 is formed especially by polar phospholipids, with the cholesteryl ester molecules from the overlying layer 3 intercalating in part. Altogether, a hydrophobic platform is thus formed for the nonpolar triglycerides 4, which form the outer boundary layer to the environment.

FIG. 1B shows the situation in the presence of 100 mM ectoine in the lacrimal fluid. The molecules in the layers 2 and 3 of the lipid layer that are adjacent to the aqueous phase are arranged in a distinctly less compact manner than in depiction a); therefore, they no longer form a continuous platform for the triglycerides 4. These therefore form small droplet-type local clusters at the boundary layer to the air. Altogether, the lipid layer is distinctly more mobile and less rigid in the presence of ectoine, and this lowers the risk of rupturing. Furthermore, the lipid layer has a thickness of approx. 200 nm at the sites of the droplet-type clusters, whereas the lipid layer in the absence of ectoine only has a thickness of approx. 20 nm.

The composition according to the invention is intended especially for local or topical application. Accordingly, the composition can be present in liquid form, for example in the form of eye drops. An aqueous solution is generally involved. The composition can, for example, be isotonic, hypotonic or hypertonic. In addition, other administration forms are, however, also conceivable, for example creams or gels.

Possible pharmacologically compatible salts of ectoine/hydroxyectoine are the alkali metal or alkaline earth metal salts, more particularly the salts of potassium, sodium, magnesium and calcium, but Iso salts with organic bases, such as with nontoxic aliphatic or aromatic amines for example.

By reacting the carboxyl group of ectoine/hydroxyectoine with alcohols or amines, it is possible to obtain corresponding esters or amides, which are likewise usable according to the invention. In the case of an amide, the amide function can in turn have saturated or unsaturated, linear or branched alkyl groups. In the case of hydroxyectoine, the hydroxyl group can also be reacted with a carboxylic acid of differing chain length to form a corresponding ester.

The composition can contain customary excipients, for example vehicles, preservatives, bactericides, solubilizers, vitamins, stabilizers, substances for preventing foaming, osmotically active substances, dyes, surface-active substances, emulsifiers, humectant substances and the like.

Preservatives are, for example, thimerosal, organic mercury compounds such as phenylmercury, benzalkonium chloride, chlorhexidine, benzyl alcohol, glucose, ethanol and quaternary ammonium salts.

Especially advantageous is the addition of viscosity-increasing agents. Specifically, it has been found that, surprisingly, an increased viscosity additionally supports the stabilization of the tear film and provides better results than the use of ectoine/hydroxyectoine without a viscosity-increasing additive. The reason for this is presumably because the ectoine/hydroxyectoine is kept longer on the eye and can therefore contribute to the stabilization of the tear film over a longer period. Furthermore, the exposure is more comfortable for the patient.

Examples of viscosity-increasing agents are cellulose ethers such as hydroxyethylcellulose, carboxymethylcellulose, hydroxypropylmethylcellulose, methylpropylcellulose, methylcellulose, methylethylcellulose, ethylcellulose, hydroxyethylmethylcellulose, hydroxypropylcellulose, ethylhydroxyethylcellulose. Other examples are polyethyleneglycol, polyvinyl alcohols, polyvinylpyrrolidone, glycosaminoglycans, proteoglycans, cetyl alcohol and stearyl alcohol or combinations therefrom (cetostearyl alcohol), polyacrylic acid, polymethacrylic acid, polyacrylamide, polyethers, polyimines, polyamides, alginates, xanthane, polyuronides, alginic acid, carrageenan, chondroitin sulfate, guar gum, hydroxypropyl guar gum and starch acetate.

The concentration of the viscosity-increasing agents in the composition is with preference from 0.05 to 10% by weight, preferably from 0.1 to 3% by weight. For example, concentrations within the range from 0.2 to 2.5% by weight for cellulose ethers, within the range from 0.2 to 1% by weight for polyethylene glycol, from 0.1 to 4% by weight for polyvinyl alcohol and from 0.1 to 0.3% by weight for polyacrylic acid have been found to be appropriate.

Moisturizing or humectant substances are, for example, glycerol, sorbitol, trehalose, betaine, dexpanthenol, 1,2-propyleneglycol, xylitol or other polyalcohols.

The formulations of the invention can likewise contain suitable buffer systems or other excipients for adjustment of pH in order to set and maintain a desired pH. Suitable buffer systems are citrate, phosphate, TRIS, glycine, borate, acetate. Said buffer systems can be prepared from substances such as citric acid, monosodium phosphate, disodium phosphate, glycine, boric acid, sodium tetraborate, acetic acid or sodium acetate.

The compositions can contain further active ingredients; however, it is especially also possible, and sufficient for the treatment or prevention of an eye disease, to use compositions which only contain ectoine and/or hydroxyectoine or corresponding salts, esters or amides as active ingredient.

Further active ingredients can, for example, be other compatible solutes. Here, it is possible to mention in particular di-myo-inositol phosphate (DIP), cyclic 2,3-diphosphoglycerate (cDPG), 1,1-diglycerophosphate (DGP), P-mannosylglycerate (firoin), β-mannosylglyceramide (firoin A), dimannosyl-diinositol phosphate (DMIP), glucosylglycerol, taurine, betaine, citrulline, 4,5-dihydro-2-methylimidazole-4-carboxylic acid (DHMICA) and 4,5,6,7-tetrahydro-2-methyl-1H-[1,3]-diazepine-4-S-carboxylic acid (homoectoine) and also corresponding derivatives, more particularly salts, esters or amides. Other suitable active ingredients are local anti-inflammatoirexample steroids, cyclosporine A, beta-receptor blockers.

Antibiotics can also be part of the composition. These include gentamicin, kanamycin, neomycin, tobramycin, ciprofloxacin, ofloxacin, chlortetracycline, ciprofloxacin, erythromycin, fusidic acid, lomefloxacin, levofloxacin and oxytetracycline.

Other active ingredients of natural origin can be: omega-3 fatty acids, eyebright, digitalis, euphrasia, blueberry, chamomile, mallow and aloe vera.

The concentration of ectoine/hydroxyectoine and/or corresponding salts, esters or amides can be especially within a range from 10 to 500 mM, preferably from 50 to 500 mM, particularly preferably from 100 to 500 mM or from 100 to 200 mM. These concentrations have been found to be suitable for bringing about the effect according to the invention. More particularly, the proportion of ectoine/hydroxyectoine and/or corresponding salts, esterds or amides in the composition can be within a range from 0.1 to 10% by weight. For example, it was possible to observe a good effect within a range between 0.5 and 2% by weight.

To improve the application and the shelf life of the composition according to the invention, the composition containing the active ingredient can also be encapsulated in nanostructures or be administered in the form of liposomes. This is especially advantageous when the composition does not contain any preservatives, this being preferred with respect to the intended use in the eye. Relevant methods for encapsulation are absolutely known from the prior art. The use of liposomes is also particularly advantageous because the lipids forming the membrane of the liposome, including phospholipids and fatty acids, also contribute to the formation of the meibomian film.

Furthermore, the invention also relates to a spray device which can be used to apply the composition according to the invention to the open or closed eye. The spray device has means for the atomization of the composition and has usefully an outlet opening, the size of which is matched to the size of the eye. The user holds the spray device such that the outlet opening is in front of the eye, and so the eye is wetted with the composition upon actuation of the spray device.

### Experiment 1

Meibomian lipids were obtained from the meibomian glands of four healthy volunteers. The lipids were dissolved in chloroform/methanol (1:1, v/v) and applied to an aqueous subphase (PBS, phosphate-buffered saline solution, pH 7.4, T=20° C.). After an equilibration time of 10-15 min, the monolayers were compressed at 2.9 cm²/min. The subphase was provided with different ectoine concentrations.

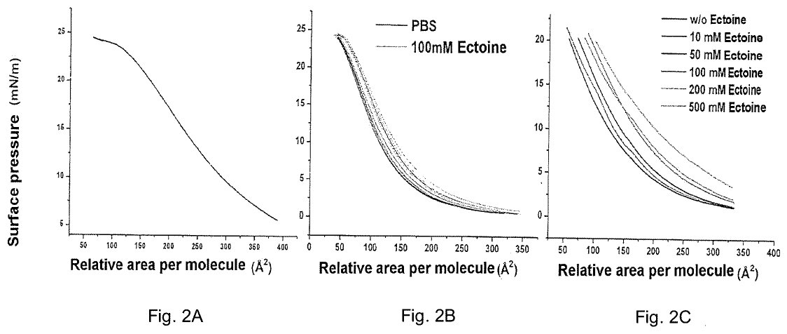

The result of the compression of the lipid film on a PBS subphase without ectoine is depicted in FIG. 2A. No phase transitions are observed up to a surface pressure of 20 mN/m. In the presence of ectoine, the isotherm shifts in the direction of a larger area per molecule, associated with a larger area occupied by the lipid head groups. Ectoine therefore enlarges the spaces between the molecules. As can be seen in FIG. 2C, the effect is concentration-dependent. FIG. 2B shows compression-expansion curves; with respect to hysteresis, no significant differences are observed.

The experiments show that the individual molecules from the lipid layer occupy a larger area in the presence of ectoine, i.e., the lipid layer becomes altogether more fluid and less rigid and therefore less inclined to rupture.

### Experiment 2

The topography of the surface of the lipid layers was examined with the aid of an atomic force microscope (atomic force microscopy, AFM). To this end, the lipid layer was transferred to a solid surface using the Langmuir-Blodgett technique; thereafter, the AFM measurements were carried out at pressures of 5 mN/m, 20 mN/m and 23.5 mN/m and 20° C. At a relatively low pressure of 5 mN/m, it was possible to observe small fibrous structures of up to approx. 5 nm in height distributed over the entire area. Layers of hydrophobic lipids such as cholesteryl esters and triglycerides might be involved here. At higher pressures of 20 mN/m, the number of fibrous structures increased.

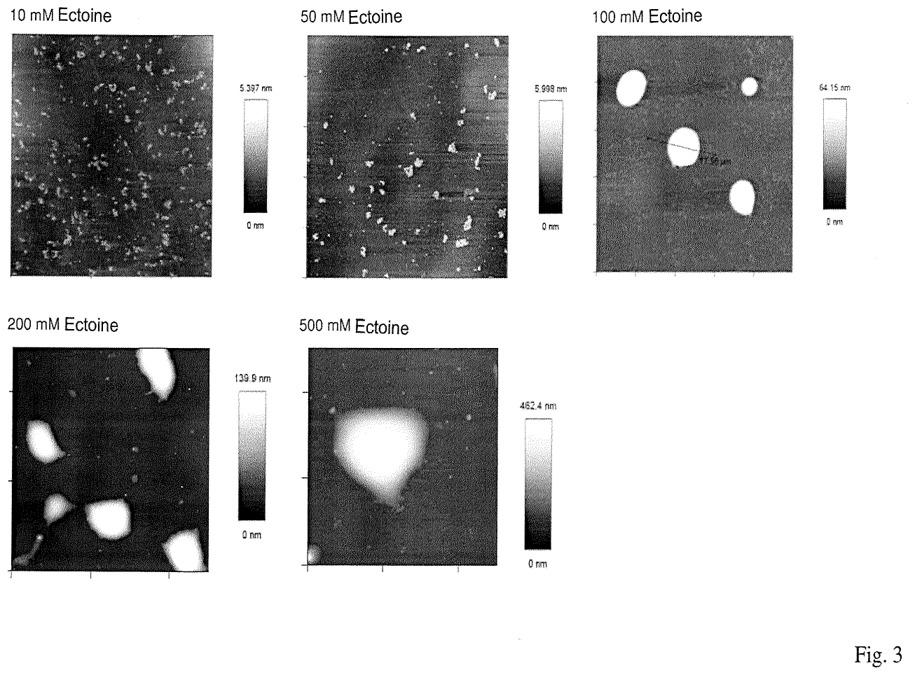

In the presence of 100 mM ectoine, droplet-type structures of up to approx. 300 nm in height were observed. At higher ectoine concentrations, the diameter of the droplet-type structures increased. In the presence too of 50 mM ectoine, it is possible to observe the formation of such structures, though still comparatively scattered and distinctly smaller. It was possible to show that the droplet-type structures are more fluid than the other regions of the lipid layer, indicative of a concentration of triglyerides which are less strongly molecularly organized than phospholipids and cholesteryl esters. The result is displayed in FIG. 3.

Altogether, the AFM examinations therefore also show that the lipid layer in the presence of ectoine is more fluid, this being associated with a reduction in the risk of rupturing in the lipid layer.

It was possible to achieve similar results with artificial meibomian films. These were generated by combining together a phospholipid (DPPC, 1,2-dipalmitoyl-sn-glycero-3-phosphocholine), a cholesteryl ester (CP, cholesteryl 3-palmitate) and a triglyceride (DPOG, 1,3-dipalmitoyl-2-oleoylglycerol) in different 10 ratios.

### Experiment 3

In one study, 64 patients suffering from a mild to moderate form of keratoconjunctivitis sicca were examined. All the patients were older than 18 years and exhibited acute symptoms (severity level 1-3 according to the Dry Eye Work Shop (DEWS); Research in dry eye: report of the Research Subcommittee of the International Dry Eye Workshop. Ocul Surf 2007; 5 (2): 179-193). The majority (72%) of the patients were female. For at least one eye in each case, the tear break-up time (TBUT), i.e., the time until dry spots reappear on the cornea after blinking, was less than 10 sec. The TBUT was used as the main parameter for the treatment assessment.

Over a period of 28 days, 34 patients were treated with an ectoine-containing composition and the other 30 patients with hyaluronic acid as reference. 59 of the patients finished the study as planned; 5 patients left prematurely.

On average, the TBUT improved within the 28-day observation period by 2.6 sec (p=0.0011) for ectoine-treated patients and by 1.1 sec (p=0.1686) for the hyaluronic acid-treated patients. Especially the patients with severe keratoconjunctivitis sicca and a TBUT<5 sec showed a stronger improvement in the case of treatment with ectoine than in the case of treatment with hyaluronic acid.

The quality of life of the patients was determined on the basis of the OSDI (ocular surface disease index). The OSDI assessment consists of 12 questions answered by the patients on a scale of 0 to 4 (0=never; 4=always). The higher the OSDI value, the stronger the impairment of the daily life of the disease. Within the observation period, it was possible to observe a decrease of 17.7 points in the case of the ectoine-treated patients and of 17.2 points in the case of the hyaluronic acid-treated patients.

Lacrimation was examined according to the Schirmer's test II. This was lower for the ectoine patients than for the hyaluronic acid patients both at the start (8.8 mm/5 min compared to 16.0 mm/5 min) and at the end (10.6 mm/5 min compared to 16.8 mm/5 min) of the treatment, it being possible to observe a more distinct improvement for the ectoine-treated patients than for the hyaluronic acid-treated patients (1.8 mm/5 min compared to 0.8 mm/5 min).

The estimation of the effectiveness of the treatment was comparable between the patient groups, but somewhat better for ectoine than for hyaluronic acid according to patients' estimates (0.9 compared to 0.6 points in the assessment). Especially the sensation of foreign bodies and the sensation of tired eyes were rated as improved following the treatment. 78.8% of the ectoine-treated patients and 71.4% of the hyaluronic acid-treated patients would continue use. Both the treatment with ectoine and the reference treatment were generally well tolerated.

### EXEMPLARY EMBODIMENTS

### Example 1

Eye drops having the following compositions are used:
0.5% by weight of ectoine
0.2% by weight of polyacrylic acid
10 0.4% by weight of NaC1
ad 100 water

### Example 2

Eye drops having the following compositions are used:
0.500% by weight of ectoine
3.950% by weight of sorbitol
0.310% by weight of hydroxyethylcellulose
0.294% by weight of trisodium citrate dihydrate
Citric acid for adjustment of pH
ad 100 water

1-12. (canceled)

13. A method for at least one of a treatment and a prophylaxis of dry eye syndrome, the method comprising:
applying a composition to an open or closed eye,
wherein the composition comprises
as an active ingredient, at least one of ectoine, hydroxyectoine and salts, esters or amides of these compounds, wherein the active ingredient is present in a range of 0.1 to 10% by weight,
a first viscosity-increasing agent which is a glycosaminoglycan,
a second viscosity increasing agent selected from the group consisting of carboxymethylcellulose, methylcellulose, methylethylcellulose, hydroxyethylcellulose, ethylcellulose, ethylhydroxyethyl cellulose and hydroxyethylmethylcellulose, and
a buffer system,
wherein the concentration of the viscosity-increasing agents in the composition is from 0.05% to 10% by weight.

14. The method according to claim 13, wherein said dry eye syndrome is a keratoconjunctivitis sicca.

15. The method according to claim 13, wherein the composition comprises an aqueous solution.

16. The method according to claim 13, wherein the at least one of ectoine, hydroxyectoine and salts, esters or amides of these compounds is present in a concentration between 10 mM and 500 mM.

17. The method according to claim 16, wherein the at least one of ectoine, hydroxyectoine and salts, esters or amides of these compounds is present in a concentration of between 100 mM and 500 mM.

18. The method according to claim 13, wherein the concentration of the viscosity-increasing agents in the composition is from 0.1 to 3% by weight.

19. The method according to claim 13, wherein the composition is an isotonic, a hypotonic, or a hypertonic composition.

20. The method according to claim 13, wherein the composition is encapsulated in nanostructures or is present in the form of liposomes.

21. The method according to claim 13, wherein the buffer system is prepared from one or more substances selected from the group consisting of citric acid, monosodium phosphate, disodium phosphate, boric acid, glycine, acetic acid and sodium acetate.

22. The method according to claim 13, wherein the glycosaminoglycan is hyaluronic acid.

23. A method for at least one of a treatment and a prophylaxis of dry eye syndrome, the method comprising:
applying an aqueous composition to an open or closed eye,
wherein the aqueous composition consists of
as an active ingredient, at least one of ectoine, hydroxyectoine and salts, esters or amides of these compounds, wherein the active ingredient is present in a range of 0.1 to 10% by weight,
a first viscosity-increasing agent which is a glycosaminoglycan,
a second viscosity increasing agent selected from the group consisting of carboxymethylcellulose, methylcellulose, methylethylcellulose, hydroxyethylcellulose, ethylcellulose, ethylhydroxyethyl cellulose and hydroxyethylmethylcellulose, and
a buffer system,
wherein the concentration of the viscosity-increasing agents in the aqueous composition is from 0.05% to 10% by weight.

24. The method according to claim 23, wherein said dry eye syndrome is a keratoconjunctivitis sicca.

25. The method according to claim 23, wherein the at least one of ectoine, hydroxyectoine and salts, esters or amides of these compounds is present in a concentration between 10 mM and 500 mM.

26. The method according to claim 23, wherein the at least one of ectoine, hydroxyectoine and salts, esters or amides of these compounds is present in a concentration of between 100 mM and 500 mM.

27. The method according to claim 26, wherein the concentration of the viscosity-increasing agents in the composition is from 0.1 to 3% by weight.

28. The method according to claim 23, wherein the composition is an isotonic, a hypotonic, or a hypertonic composition.

29. The method according to claim 23, wherein the buffer system is prepared from one or more substances selected from the group consisting of citric acid, monosodium phosphate, disodium phosphate, boric acid, glycine, acetic acid and sodium acetate.

30. The method according to claim 23, wherein the glycosaminoglycan is hyaluronic acid.

31. The method according to claim 13,
wherein the active ingredient is present in a range of 0.5 to 5.0% by weight.

32. The method according to claim 13,
wherein the active ingredient is present in a range of 0.5 to 2.0% by weight.
